(19) United States
(12) Patent Application Publication
(10) Pub. No.: US 2026/0335475 A1
(43) Pub. Date: Oct. 29, 2026

(54) Nonthermal Far Infrared and Ionizing Radiation Emitting Ceramic Composite for Therapeutic Applications and its Fabrication Method

(71) Applicant: Albert Chin-Tang WEY, Westmont, IL (US)

(72) Inventor: Albert Chin-Tang WEY, Westmont, IL (US)

(21) Appl. No.: 19/191,779

(22) Filed: Apr. 28, 2025

(51) Int. Cl. A61K 51/12; A61N 5/06; A61N 5/10

(52) U.S. Cl. CPC A61K 51/1289; A61N 5/1029; A61K 2121/00; A61N 2005/0645; A61N 2005/0656; A61N 2005/066; A61N 2005/1019

(57) ABSTRACT

This invention relates to a ceramic composite and its fabrication method, designed for therapeutic devices that treat humans or animals using nonthermal far-infrared (FIR) and low-dose ionizing radiation. The composite simultaneously emits nonthermal FIR photons (wavelengths of 3-16 μm) and ionizing radiation (>0.05 μSv/h), which enhance physiological performance, immune competence, health, and lifespan.

### BACKGROUND

### Field of Invention

This invention relates to a ceramic composite and its fabrication method, designed for therapeutic devices that treat humans or animals using nonthermal far-infrared (FIR) and low-dose ionizing radiation. The composite emits nonthermal FIR photons (wavelengths of 3-16 μm) and ionizing radiation (>0.05 μSv/h), which enhance physiological performance, immune competence, health, and lifespan.

### Description of Prior Art

Hormesis is the stimulation of any system by low doses of any agent. Radiation hormesis is the hypothesis that ionizing radiation is benign at low levels of exposure and can be beneficial. This contrasts with the Linear-No-Threshold (LNT) model which posits that the negative health effects of ionizing radiation are proportional to the dose and there is no “safe dose” (threshold). While most authorities agree that the LNT model is most appropriate, numerous studies have proposed radiobiological hormesis, suggesting that radiation levels of 100 mSv/y (milli-Sieverts per year), or equivalently 11.4 μSv/h (micro-Sieverts per hour), may be positive or at least neutral to health.

While studying radiation hormesis theory, the present inventor found that combining low-dose ionizing radiation with far-infrared (FIR) radiation may synergistically enhance health benefits. Such radiation supplementation may potentially influence the hydrolysis of Adenosine Triphosphate (ATP), serving as an example of its biological effects. Under the combined effects of FIR radiation and low-dose ionizing radiation, ATP hydrolysis effectively drives essential processes in living cells, such as muscle contraction, nerve impulse transmission, and chemical synthesis. The increased energy availability from ATP enables cells to function more efficiently, rejuvenate, and repair damage.

As discussed above, simultaneous exposure to FIR radiation and low-dose ionizing radiation presents unique opportunities to enhance human health. This discovery highlights the need for a reliable and consistent radiation source capable of emitting both FIR radiation and low-dose ionizing radiation simultaneously, which led to the invention by the present inventor (U.S. Pat Appl. US-20220032086). However, as the research progressed, the present inventor uncovered a common misconception about far-infrared radiation.

It is explained in chemistry textbooks that FIR photons within the 3-16 μm wavelength range can be absorbed by molecules, leading to vibrations at corresponding frequencies. These molecular vibrations may increase the internal energy of reactants, which in turn can reduce the activation barrier and enhance chemical reaction rates, as described by the Arrhenius equation. FIR radiation induces positive biological effects in the body, primarily through improving the chemical reactions that underpin most bioeffects.

Additionally, it is well established that all materials emit FIR radiation within the 3-1000 μm wavelength spectrum at room temperature, as part of their thermal radiation, which is characterized as Blackbody radiation. There is a common error in prior art regarding the utilization of the “3-16 μm” segment of the thermal radiation wavelength spectrum in applications that requires specific 3-16 μm wavelength photons.

In light of this, describing thermal radiation as being limited to the wavelength range of “3-16 μm” is misleading. Theoretically, blackbody radiation (thermal radiation) follows a continuous wavelength spectrum, known as the Planck spectrum, which is determined solely by the temperature of the emitting body. The segment of 3-16 μm is inherently part of the broader spectrum, ranging from 3 μm to 1000 μm, and cannot be considered in isolation. The absorption of thermal radiation serves as radiative heat transfer to molecules in condensed matter (solid and liquid), transforming into heat motion that generates a combination of electronic, molecular, and lattice oscillations. Thermal radiation spans the entire 3-1000 μm spectrum as a unified whole, making any attempts to divide it into distinct subdivisions conceptually flawed. This has been a recurring error in prior art.

Thermal radiation is a type of electromagnetic radiation that includes far-infrared radiation at room temperature. Quantitatively, emissivity (ε) represents the ratio of radiant energy emitted by a surface to the energy emitted by a blackbody at the same temperature, as defined by the Stefan-Boltzmann law. The emissivity value ranges from 0 to 1 (0<ε<1). Real objects always have emissivity less than 1.0 and emit radiation at proportionally lower rates. For example, materials such as fabric, glass, charcoal, concrete, porcelain, rubber, and sand typically exhibit emissivity between 0.80 and 0.98.

An important consideration in conventional FIR-emitting composite design is achieving an emissivity as close to 1.0 as possible. While individual inventors often pursue unique approaches to creating FIR composites, most prior art inventions have achieved emissivities in the range of 0.80-0.95, typically treating all constituent oxides for thermal radiation and without distinguishing their roles as FIR-photon-emitting oxides (for examples, U.S. Pat. Nos. 8,285,391, 9,308,388, and 9,962,441).

To meet the need for a nonthermal FIR-photon-emitting source, the present inventor created a ceramic composite with at least 10 wt. % silicon oxide and aluminum oxide, and at least 3 wt. % of chromium oxide or iron oxide. This mixture is calcined to induce at least one phase transformation, resulting in the ceramic composite comprising at least one FIR luminescence center—either an octahedral complex or a tetrahedral complex—under the influence of an electrostatic crystal field surrounding these complexes. Consequently, the ceramic composite emits nonthermal FIR radiation within the 3-16 μm wavelength spectrum (U.S. Pat Appl. US-20230322627).

Meanwhile, incorporating radioactive material into FIR-emitting ceramic is not a novel concept. Previous inventions (for example: U.S. Pat. Nos. 6,890,457, 7,395,554, 7,637,858, Patent Appl. 20120175526, and WO 2018/096651) have utilized FIR-emitting ceramic powders combined with radioactive mineral powders for various applications that involve both ionizing radiation and FIR thermal radiation. It seems intuitive that combining the findings disclosed in Patent Application US-20230322627 with previous inventions could serve as a basis for the development of a ceramic composite capable of emitting nonthermal FIR-photons and low-dose ionizing radiation.

However, while researching the integration of the two inventions (US-20220032086 and US-20230322627, both of which are incorporated by reference in their entirety), the present inventor observed that more than an octahedral complex or a tetrahedral complex, as previously taught, may be necessary when incorporating a radioactive isotope (impurity) into the polycrystalline structure.

After extensive investigation, the present inventor determined that a polymerized silicate structure—preferably a phyllosilicate—constructed with distorted octahedral compounds is essential. The phyllosilicate structure would consist of alternating layers of a silica tetrahedral sheet and an alumina octahedral sheet and have charge-balancing cations and radioactive elements uniformly distributed within the interstitial layer.

Furthermore, during calcination through a predetermined process, Al³⁺ ions in the octahedral site are substituted with either Cr³⁺ or Fe³⁺, forming a distorted octahedral compound. This configuration must be further processed to create a “strong-field, low-spin” environment within the phyllosilicate structure, based on Crystal Field Theory (CFT). Without the “strong field, low-spin” condition, the electron transitions in d-orbitals of Cr³⁺ or Fe³⁺ octahedral site will be forbidden by the Spin Selection Rule, and no photons can be generated.

The ceramic composite described in this invention incorporates a polymerized silicate framework featuring distorted octahedral compounds. These compounds are deliberately designed to achieve a “strong-field, low-spin” condition through precise selection of constituent materials and optimized processing techniques. This unique configuration enables the ceramic composite to emit nonthermal photons within the 3-16 μm wavelength range while simultaneously producing low-dose ionizing radiation. This dual functionality represents a novel innovation not addressed by prior art.

### Objects and Advantages

Accordingly, one objective of this invention is to develop a ceramic composite capable of simultaneously emitting nonthermal FIR photons within the 3-16 μm wavelength spectrum and ionizing radiation at a specific dose rate exceeding 0.05 μSv/h.

Another objective of the present invention is to develop a ceramic composite designed for integration into a therapeutic device. This device would leverage the principles of hormesis to effectively improve the health conditions of the human or animal body.

Additionally, another objective of the present invention is to develop a simple, effective, user-friendly, and maintenance-free therapeutic device that is flexible enough to be attached to any part of the human or animal body requiring treatment.

These objectives are achieved through a ceramic composite that simultaneously emits nonthermal far-infrared photons within the 3-16 μm wavelength spectrum and ionizing radiation at a specific dose rate exceeding 0.05 μSv/h. This ceramic composite is primarily composed of a polymerized silicate structure, featuring distorted octahedral compounds, formed from a mixture of selected oxide or mineral powders through a predetermined calcination process. The composite can be secured using a flexible attachment and positioned near the body part requiring treatment.

Other objects, features, and advantages of the present invention will hereinafter become apparent to those skilled in the art from the following description.

### SUMMARY OF THE INVENTION

In accordance with the present invention, a ceramic composite is formed from a mixture of powders of selected oxides including a radioactive isotope. This mixture is calcined along with bonding agents through a predetermined calcination process, resulting in the formation of the composite. The ceramic composite emits nonthermal far-infrared photons in the 3-16 μm wavelength range and ionizing radiation at a dose rate exceeding 0.05 μSv/h. These emissions may contribute to improving specific health conditions in humans or animals.

### BRIEF DESCRIPTION OF THE DRAWINGS

FIG. 1 is a view of a phyllosilicate structure of the present invention, consisting of alternating layers of two sheets: a silica tetrahedral sheet and an alumina octahedral sheet. Cations and radioactive elements are uniformly distributed within the interstitial layer between phyllosilicate sheets.

FIG. 2 is a top perspective view of a first embodiment of the present invention showing multiple ceramic composites are mounted on a flexible substrate for attaching to a body part to be treated, wherein each of ceramic composites has a concave surface facing the body.

FIG. 3 is a view of a second embodiment, showing a ceramic composite in the form of a sphere.

FIG. 4 is a view of a third embodiment, showing a ceramic composite in the form of a circular plate.

FIG. 5 is a view of a fourth embodiment, showing a ceramic composite in the form of a rectangular plate.

FIG. 6 is a view of a fifth embodiment, showing a ceramic composite in the form of a rod or wire.

FIG. 7 is a view of a sixth embodiment, showing a rectangular base material coated with the ceramic powders of the present invention.

FIG. 8 is a view of a seventh embodiment, showing a wire or fiber coated with the ceramic powders of the present invention.

### REFERENCE NUMERALS IN DRAWINGS

11 ceramic composite
12 substrate
13 base material
14 fiber or wire
21 oxygen atom
22 silicon atom
23 aluminum atom
24 charge-balancing cation
25 radioactive isotope
31 ceramic particles

### DETAILED DESCRIPTION OF THE INVENTION

A therapeutic device, comprising the ceramic composite of present invention, is designed to simultaneously emit nonthermal FIR photons within the 3-16 μm wavelength spectrum and ionizing radiation at a dose rate exceeding 0.05 μSv/h. In practice, each ceramic composite is tailored to emit ionizing radiation at a dose rate ranging from 0.05 to 0.5 μSv/h, allowing an assembly of multiple composites to achieve a specified dose rate finely calibrated within the 0.05-5.7 μSv/h range to promote beneficial radiation hormesis effects.

The ceramic composite is composed of a mixture of powders from three sets. The first set comprises at least 8 wt. % silicon oxide (SiO₂) and at least 2 wt. % aluminum oxide (Al₂O₃). The second set includes at least 2 wt. % chromium oxide (Cr₂O₃) or iron oxide (Fe₂O₃). The third component is a powdered substance containing at least 0.1 wt. % of radioactive isotope. This mixture is calcined through a predetermined process to produce the ceramic composite. In practical applications, additional charge-balancing cation dopants, such as potassium (K), sodium (Na) and calcium (Ca), can be incorporated to enhance the crystal field properties.

The composite is designed and processed to comprise a polycrystalline structure, including at least one polymerized silicate, constructed with distorted octahedral compounds. The polymerized silicate may range from simple chain silicates and sheet silicates to complex framework silicates, while a phyllosilicate structure is preferred.

The phyllosilicate structure consists of alternating layers of two sheets: a silica tetrahedral sheet and an alumina octahedral sheet. The silica tetrahedral sheet is composed of silicon-oxygen tetrahedra, where each silicon atom is bonded to four oxygen atoms (SiO₄). The alumina octahedral sheet contains aluminum atoms coordinated with six oxygen atoms in an octahedral arrangement (AlO₆). Charge-balancing cations and radioactive elements are uniformly distributed within the interstitial layer.

During calcination, Al³⁺ ions in the octahedral site are substituted with either Cr³⁺ or Fe³⁺, leading to the formation of a distorted octahedral compound. The resulting composition is processed to create a strong-field, low-spin environment within the phyllosilicate structure, as described by Crystal Field Theory (CFT). Each transition metal coordinate complex, [CrO₆]³⁺ or [FeO₆]³⁺ becomes a persistent luminescence site for 3-16 μm wavelength photons through electron transitions between d-orbital levels. Consequently, the ceramic composite emits nonthermal photons in the 3-16 μm wavelength range while simultaneously generating low-dose ionizing radiation.

The need for a mixture comprising at least 8 wt. % silicon oxide (SiO₂), at least 2 wt. % aluminum oxide (Al₂O₃), and at least 2 wt. % chromium oxide (Cr₂O₃) or iron oxide (Fe₂O₃) arises from the minimum requirements for forming an effective phyllosilicate structure.

Phyllosilicates are characterized by their sheet-like structures. It starts with the right materials (SiO₂ and Al₂O₃) by combining these materials in specific ratios to achieve the required composition as precursors. For this reason, in some embodiments, powders of phyllosilicate minerals in aluminosilicate group, such as kaolinite (Al₂O₃·2SiO₂·2H₂O), montmorillonite (Na, Ca)(Al, Mg)₂(Si₄O₁₀)(OH)₂, and illite (K) (Al, Mg, Fe)₂(Si, Al)₄O₁₀(OH)₂ may be added to the mixture serving as precursors.

This is followed by heating the mixture, sometimes under pressure, allowing the materials to crystallize into the layered structure that defines phyllosilicates. The time and conditions required depend on the desired properties of the final composite and must be meticulously calculated and predefined.

The phyllosilicate structure can adopt either a “T-O” or “T-O-T” layered configuration, where T represents a tetrahedron and O represents an octahedron. Considering the potential substitution of Al³⁺ by Cr³⁺ or Fe³⁺, it is preferred to maintain the weight ratio of SiO₂ to that of the sum of Al₂O₃, Cr₂O₃, and Fe₂O₃ between 1 and 3. For instance, in one embodiment, the mixture consists of approximately 50 wt. % SiO₂, 20 wt. % Al₂O₃, 16 wt. % Fe₂O₃, and 4 wt. % Cr₂O₃, yielding a ratio of 1.25. The remaining wt. % of the mixture consists of oxides of charge-balancing cations (e.g., K₂O , Na₂O, or CaO) and minerals containing radioactive isotopes.

Sometimes, it may be beneficial to use natural materials like minerals instead of pure oxides. For instance, in another embodiment, the oxides of SiO₂ and Al₂O₃ are proportionally substituted with kaolinite and quartz, whereas Cr₂O₃, and Fe₂O₃ are replaced by hematite (Fe₂O₃), magnetite (FeO·Fe₂O₃), or chromite (FeO·Cr₂O₃). Minerals can be more cost-effective and environmentally friendly for some uses, making them a good alternative in various scenarios. Nonetheless, using pure oxides instead of minerals offers several advantages, primarily in terms of control, consistency, and application-specific performance. It all depends on the balance of precision and practicality for any specific use case.

In the calcination process, Cr³⁺ and Fe³⁺ substitute for Al³⁺ in the octahedral sites exclusively via a charge-coupled substitution mechanism. The phase transition begins at approximately 950 °C, but calcination at temperatures exceeding 1150 °C, preferably above 1200 °C, is critical to form the essential distorted octahedral compound. As temperature increases and dopant (Cr³⁺ and Fe³⁺) concentration rises, the lattice constant decreases due to densification and structural rearrangements. This effect is attributed to the distinct ionic radii of Cr³⁺ (0.615 Å) and Fe³⁺ (0.550 Å) compared to Al³⁺ (0.535 Å) in octahedral sites. Furthermore, the phenomenon is highly sensitive to local disorder and substitutional inhomogeneities, influencing the material's structural evolution.

Due to Fe—O bond shortening in the electronic structure of [FeO₆]³⁺ compounds, which increases electron repulsion, the Fe³⁺ orbital energy levels in octahedral sites transition from high-spin (HS) to low-spin (LS) states. The crystal field splitting energy of LS Fe³⁺ is approximately 22,200 cm⁻¹, derived from the energy difference (Δₒ) between the centers of the t_{2g} and e_{g} bands. This Δₒ value is calculated as 2.70 eV (21,800 cm⁻¹), characteristic of octahedrally-coordinated LS Fe³⁺. The LS Fe³⁺configuration is crucial for establishing the persistent luminescence mechanism, enabling 3-16 μm photon emission within the ceramic composite of present invention.

Based on Molecular Orbital (MO) theory of transition metal (TM) complexes, the characteristics of transition metal-ligand (M—L) bonds become well-defined by analyzing the molecular orbitals formed through the overlap of metal 3d orbitals with ligand orbitals in an octahedral complex [ML₆]. Crystal Field Theory (CFT) complements this by describing the energy splitting of the d orbitals and the electronic structure of TM-complexes, particularly their optical and magnetic properties.

There are five degenerate d-orbitals in a free transition metal ion. When a TM-complex has an octahedral coordination sphere, with six ligands positioned along the x, y, and z axes, two specific d orbitals interact strongly with the ligands: dₓ_{²}_{-y}_{²}, which lies along the x- and y-axes, and d_{z}_{²}, aligned along the z-axis. These strong bonding interactions raise the energy levels of these orbitals. Together, they form the “e_{g}” set of orbitals, a term derived from the mathematical symmetry of the octahedral field.

In contrast, the other three d orbitals (d_{xy}, d_{xz} and d_{yz}) are oriented between the bond axes of the donor ligands, rather than aligning directly with them. As a result, these orbitals interact less strongly with the donor electrons and are collectively referred to as the “t_{2g}” set of orbitals.

An octahedral arrangement of six negatively charged ligands around a TM ion causes the five degenerate d orbitals to split into two sets with different energies: e_{g} (higher energy) and t_{2g} (lower energy). This phenomenon is known as Crystal Field Splitting. While the total energy of the d orbitals remains conserved, the splitting redistributes their energies, which can affect the energy of the electrons depending on their arrangement among the e_{g} and t_{2g} orbitals.

The orbital degeneracy of e_{g} and t_{2g} sets of orbitals may further be lifted by the coupling to the lattice, which is called the Jahn-Teller (J-T) effect. In a so-called “Z-in J-T distortion,” the energies of d-orbitals with z factor (i.e., d_{z}_{²}, d_{xz} and d_{yz}) are increased since the bonds along the z-axis are shortened.

The building block of the planned FIR-luminescence centers in present invention is [FeO₆]³⁺, which consists of an iron-III ion (Fe³⁺) surrounded by six oxygen anions (O⁻). The Fe³⁺ ion uses 6 orbitals from the 4s, 4p and 4d levels of iron atom (sp³d² hybrid orbitals) to accept lone pairs electrons from the oxygen molecules for bonding with oxygen, forming [FeO₆]³⁺.

The FIR-luminescence is based on the Fe³⁺ ion, which possesses a 3d⁵ electronic configuration (indicating 5 electrons in the 3d-orbitals) located within an octahedral site. The ceramic composite's unit cell is calcined into a crystal geometry inducing a “Z-in J-T distortion.” This process lifts the degeneracy of the orbitals in the t_{2g} group, lowering the energy of the d_{xy} orbital relative to the d_{xz}, d_{yz} orbitals, with an energy difference δ₂. The difference, δ₂, is designed to be 100-315 meV, corresponding to λ≈4-12 μm, which covers a photoluminescence profile in the 3-16 μm band. Note: E(eV)=1.2398/λ(μm); where λ (μm) is wavelength of FIR photon.

Consequently, in the “strong field, low spin” scenario, the five electrons are arranged such that two electrons occupy the lower d_{xy} orbital while the remaining three fill the upper d_{xz}, d_{yz} orbitals.

When the ceramic composite absorbs thermal radiation from the ambience, the incident thermal radiation is converted into phonons (the wave of lattice vibrations). On one hand, the lattice vibrations in the composite will generate heat to increase the body temperature and emit thermal radiation to reach and maintain thermodynamic equilibrium with the surrounding. On the other hand, the lattice vibrations will trigger dynamic J-T distortions in the TM-ion octahedrons, swinging between “Z-in” and “Z-out” J-T distortions.

According to the La Porte Selection Rule, d-to-d transitions are forbidden in centrosymmetric complexes such as octahedral complexes. However, when the central transition metal (TM) ion is placed in a distorted field, the octahedral geometry can deviate from centrosymmetry due to molecular vibrations. These odd vibrations of the surrounding ligands temporarily create a distorted field, lasting long enough (on the order of 10⁻¹⁵ seconds) for electronic transitions to occur, as described by the Franck-Condon Principle.

As described previously, in the “Z-in” configuration, two electrons occupy the lower-energy d_{xy} orbital, while the remaining three electrons fill the higher-energy d_{xz}, d_{yz} orbitals. When switched to the “Z-out” configuration, the energy levels of the d_{xy} orbital and the d_{xz}, d_{yz} orbitals reverse, with the d_{xz} and d_{yz} orbitals now having lower energy than the d_{xy} orbital. One of the two electrons in the d_{xy} orbital transitions to fill the vacancy in the d_{xz}, d_{yz} orbitals, emitting a photon in the wavelength range of 3-16 μm, corresponding to the energy difference δ₂. The lattice vibration periods are on the order of 10⁻¹³ seconds, while the electronic transitions occur on the order of 10⁻¹⁶ seconds. This disparity satisfies the Franck-Condon Principle, as the rapid electronic transitions occur without significant changes to the nuclear configuration, ensuring vertical transitions in the energy diagram.

Over time, the system switches back from the “Z-out” to the “Z-in” configuration. During this transition, one electron moves from the d_{xz}, d_{yz} orbitals (higher energy) to fill the vacancy in the d_{xy} orbital (lower energy), emitting a photon in the wavelength range of 3-16 μm. This process continues cyclically, with the system alternating between the “Z-in” and “Z-out” configurations. Consequently, the FIR-photon emissions from the ceramic composite described in the present invention are spontaneous and continuous, persisting indefinitely as long as the conditions for emission are maintained.

This brings up an important point: the ceramic composite described in the present invention must undergo processing at temperatures above 1200 °C to establish a “strong field, low spin” environment. This condition is essential for the building blocks of the intended far-infrared (FIR) luminescence centers, [FeO₆]³⁺.

Fe³⁺ exhibits a 3d⁵ electronic configuration, containing five d-electrons that singly occupy the degenerate 3d orbitals. The spins align parallel, following Hund's rule, which states that the lowest energy state corresponds to the highest spin angular momentum. The pairing energy P is the energy penalty for putting two electrons in the same orbital, resulting from the electrostatic repulsion between electrons. For 3d transition metal elements, a typical value of P is about 15,000 cm⁻¹ (or 1.86 eV). The key point here is that a complex will be “strong field, low spin” if Δₒ>P, and “weak field, high spin” if Δₒ<P.

When the ceramic composite is processed at a temperature below 1150 °C, it results in a “weak field” condition with Δₒ <P. Under these conditions, the five unpaired electrons in the Fe³⁺ complex will still singly occupy the five 3d orbitals, with an orbital configuration of (t_{2g})³(e_{g})², even though the degeneracy is lifted. In this case, even if phonon-activated dynamic J-T distortions occur and the energy levels of d_{xy} orbital and the d_{xz}, d_{yz} orbitals reverse, no photons will be generated because the electrons prefer single occupancy in each orbital, preventing any electron transitions.

Similar conditions but different scenarios also apply to [CrO₆]³⁺ complexes. Cr³⁺ exhibits a 3d³ electronic configuration within the octahedral coordination environment. For a calcination below 1150 °C, the three unpaired electrons in the Cr³⁺ complex continue to singly occupy the three t_{2g} orbitals, resulting in an orbital configuration of (t_{2g})³(e_{g})⁰ upon lifting the degeneracy. This indicates that the Cr³⁺ complex has three d-electrons, which independently fill the three orbitals (d_{xy}, d_{xz} and d_{yz}). Consequently, even if phonon-activated dynamic J-T distortions cause the energy levels of d_{xy} orbital and the d_{xz}, d_{yz} orbitals to reverse, electron (spin) transitions remain forbidden by the Spin Selection rule. No photons will be generated.

A spin transition within the band at 17,000-22,000 cm⁻¹ is characteristic of octahedrally-coordinated low-spin (LS) Cr³⁺. When the ceramic composite is processed at temperatures above 1200 °C, the crystal field splitting energy difference is about 17,500 cm⁻¹ (Δₒ=2.17 eV), corresponding to blue light with a wavelength of 571 nm. Absorption of blue light can then excite an electron from the t_{2g} orbitals to the e_{g} orbital, resulting in a (t_{2g})²(e_{g})¹ configuration. In the “Z-in” condition, two electrons remain in the t_{2g} orbitals, with one occupying the d_{xy} orbital (lower energy) and the other occupying either the d_{xz}, d_{yz} orbital (higher energy). When the configuration shifts to “Z-out,” the orbitals invert, allowing the electron in the d_{xy} orbital (now higher energy) to transition to the d_{xz}, d_{yz} orbitals (lower energy), accompanied by the release of a photon.

That said, it is critical to calcine the mixture of selected powders through a predetermined process to achieve the designed polymerized silicate structure with distorted octahedral compounds in the “strong field, low spin” state, enabling the implementation of FIR luminescence mechanism.

Calcination is an effective thermal process that removes porosity from compacted powder particles, forming a solid mass. During this process, materials diffuse to fill pores at grain boundaries, facilitated by high temperatures. The driving force and kinetics of grain growth are primarily influenced by particle size and grain size distribution. Pores shrink as material diffuses to fill the empty spaces, creating boundary interfaces. In general, temperature increases the rate of all calcination mechanisms. Since grain growth is often controlled by surface diffusion, higher temperatures lead to greater densification compared to grain growth. This enhances the contact area between grains by removing pores, thereby promoting ion diffusion and dispersion at grain boundaries.

Thus, the heating parameters of the calcination process should be tailored to the conditions that promote silicon oxide polymerization and its sharing of oxygen atoms with aluminum oxide. This interaction leads to transformation and recrystallization, forming a phyllosilicate structure. Concurrently, doped ions such as Fe³⁺ from iron oxide and Cr³⁺ from chromium oxide can migrate freely within the crystal lattice. These ions may substitute Al³⁺in the octahedral sites of the phyllosilicate structure, resulting in the formation of distorted octahedral compounds, as planned to serve as FIR persistent luminescence centers in present invention.

It is also important to consider that radioactive isotope (such as uranium, thorium, potassium, cobalt, or radium), along with other cation-doping metal ions (e.g., Li⁺, Na⁺, K⁺, Mg²⁺, and Ca²⁺), may freely relocate within the crystal system during recrystallization, uniformly distributing within the interstitial layers. This process facilitates the formation of a grid of localized electric fields, creating a “strong field” situation that significantly influences the proposed electron transitions between d-orbitals within the same t_{2g} group.

Phase transformations and crystallization during calcination are influenced by various factors, including temperature, pressure, composition, time, crystallography, and the heating or cooling rate. Among these, temperature plays a critical role as the primary factor determining the driving force behind phase transformations. Additionally, the duration for which the temperature is maintained significantly affects the kinetics of the phase change, enabling the formation of equilibrium phases. While there are various approaches, a calcination process at 1200 °C or higher for at least 2 hours is generally preferred, although it strongly depends on the composition and particle sizes. In some cases, a lower calcining temperature may be used, compensated with longer duration of calcination.

As previously described, the FIR persistent luminescence mechanism relies on distorted octahedral compounds, specifically [FeO₆]³⁺ or [CrO₆]³⁺ complexes under a strong crystal field, combined with a radioactive isotope embedded in the interstitial layer. This unique configuration enables the ceramic composite to simultaneously emit FIR photons within the 3-16 μm wavelength range and produce ionizing radiation at a specific dose rate, offering innovative functionalities not disclosed by prior technologies.

It is worth noting that the Occupational Safety and Health Administration (OSHA) and the International Commission on Radiological Protection (ICRP) may have somewhat different radiation standards, but both concur with an annual dose limit of 50 mSv for the entire body. The guideline specifies a maximum safety dose rate of 5.7 μSv/h, corresponding to continuous exposure over 1 year (8,760 hours).

In other words, radiation hormesis theory suggests that a beneficial dose-response effect might occur within a range of low-dose levels, defined by the background radiation dose rate (1 mSv/y) and the safety maximum dose limit (50 mSv/y), or equivalently, 0.1-5.7 μSv/h. Thus, the objective of the present invention is to produce a ceramic composite capable of emitting ionizing radiation at a dose rate greater than 0.05 μSv/h but less than 5.7 μSv/h.

To achieve this goal, the ceramic composition of the present invention includes a third set of powdered substances containing one or more radioactive isotopes of uranium (U), thorium (Th), potassium (K), cobalt (Co), or radium (Ra). Among them, thorium is preferred because it exists in a natural mineral, Monazite-(Ce, La, Nd, Th)PO₄.

In Monazite-(Ce), the thorium content may vary, occasionally reaching 20-30%. Uranium is present as a trace element in certain specimens, contributing to their minimal radioactivity. For production consistency, only Monazite-(Ce) containing 6-12% thorium is utilized in the present invention. The effective ionizing radiation dosage, as a function of the weight percentage (wt. %) of Monazite-(Ce), would be experimentally determined. Based on the known wt. % of Monazite-(Ce), the ceramic composite can be designed to emit ionizing radiation within a dosage range of 0.05-5.7 μSv/h. In one embodiment, 2 wt. % of monazite is used to produce a dosage of 0.2 μSv/h. Although the ionizing dosage is minimal, the radioactive properties of the material necessitate careful handling and strict adherence to established safety protocols during use.

Additionally, in the Monazite-(Ce) system, the crystal structures of Ce oxides transition from monoclinic to cubic as the calcination temperature exceeds 1100 °C Calcination at temperatures above 1200 °C, as proposed in the present invention, may facilitate uniform mixing with other cation-doping metal structures in the interstitial layer, significantly influencing the stability and radioactivity of the resulting ceramic composite with respect to the intended ionizing radiation.

The ceramic composite of the present invention can be used as it is, or ground into submicron particles. The preferred grain size of the particles is greater than 50 nanometers (nm). These finely ground ceramic particles can be embedded in an elastomer material, such as synthetic resin, polyvinyl chloride, silicone rubber, polyurethane, polystyrene, polyethylene, or similar substances. This process can form shaped articles, including foam, pads, sheets, mats, beads, or plates. Alternatively, the particles can be deposited on a base material, including plastic, rubber, metal, wood, or ceramic. They may also be coated on fiber or cloth to expand their potential applications.

Nonetheless, it is worth noting here that, when comparing the ceramic powder and polycrystalline pressed pellets of the same composition, the Raman spectra of the powders exhibit all the main features of the spectra for ceramics based on the corresponding systems. However, all bands broaden and shift towards higher wavenumbers. Meanwhile, the bulk pellet form demonstrates approximately five times greater radiation strength compared to the powder form.

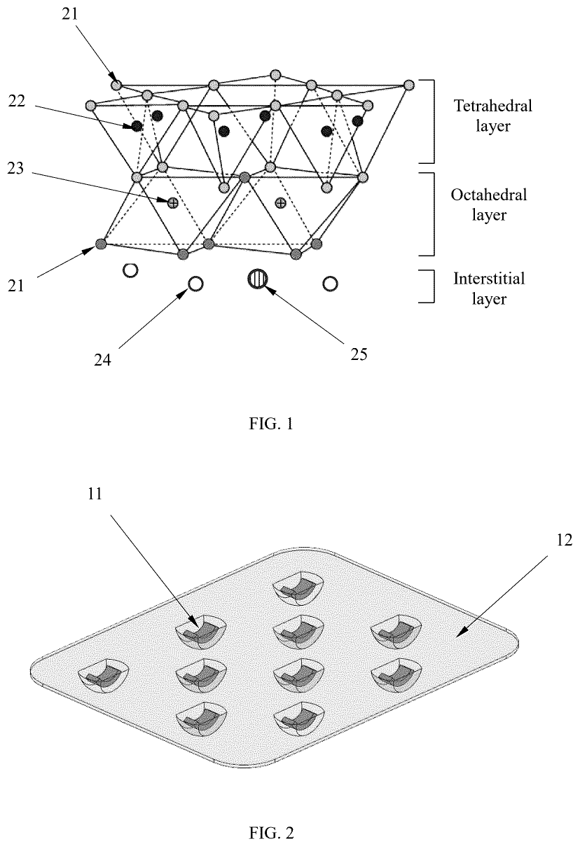

Following the preparation and process as described above, a ceramic composite of present invention can be made, capable of emitting 3-16 μm wavelength nonthermal photons and ionizing radiation at a dose rate greater than 0.05 μSv/h. FIG. 1 illustrates the key architecture of the FIR luminescence mechanism in the present invention, featuring a polymerized silicate structure, particularly phyllosilicate, with distorted octahedral compounds.

Phyllosilicates, also known as sheet silicates, have a distinctive structure comprising layered arrangements of silicon-oxygen tetrahedra and aluminum-oxygen octahedra. In a simple “T-O” (tetrahedral-octahedral) structure, each tetrahedron (SiO₄) contains a silicon atom (22) sharing three of its four oxygen atoms (21) with adjacent tetrahedra, forming a two-dimensional sheet. The tetrahedral silica (SiO₄) sheet is connected through oxygen atoms to an octahedral alumina (AlO₆) sheet. The alumina octahedral sheet consists of aluminum ions (23) coordinated with six oxygen atoms (21) in an octahedral geometry. These sheets are stacked and stabilized by layers of cations (24), such as potassium, sodium, and calcium, which balance the charge and contribute to the crystal field. Furthermore, radioactive isotopes (25) are processed to distribute uniformly within the interstitial layers. During calcination, aluminum ions (Al³⁺) (23) at the octahedral sites are substituted by chromium ions (Cr³⁺) or iron ions (Fe³⁺), resulting in the formation of distorted octahedral compounds.

The phyllosilicates can also adopt “T-O-T” (tetrahedral-octahedral-tetrahedral) structures. Beyond phyllosilicates, the polymerized silicates in the present invention may include either simple chain silicates or more complex framework silicates, provided they incorporate the necessary distorted octahedral compounds.

FIG. 2 illustrates a preferred embodiment of the present invention, where multiple ceramic composites (11) are embedded in a substrate. The substrate (12) can be made of silicone (polydimethylsiloxane), zinc sulfide, sodium chloride, potassium bromide, or similar materials. It takes the form of a substantially flat sheet that features a series of pockets—curved protrusions designed to house ceramic composites (11). In this embodiment, all pockets are uniformly dimensioned to accommodate ceramic composites of identical sizes. However, in other embodiments, the pockets may vary in size or shape to suit specific applications or arrangements involving different ceramic composites. The assembly may be equipped with a flexible mechanism for securing the composite to a body part requiring treatment.

Forming the ceramic composite (11) with a concave shape, as shown in FIG. 2, could be advantageous. Ceramic composites emit FIR and ionizing radiation uniformly in all directions. A concave surface, however, may focus the radiation toward a specific region or point, thereby enhancing its penetration into the body with less attenuation compared to a flat surface. This focused radiation could provide greater therapeutic effects relative to flat ceramics of similar mass and dimensions. Examples of potential concave shapes include hemispherical, bowl-shaped, or partial cylindrical designs. To optimize its therapeutic impact, the concave surface of the composite should be positioned to face the body part being treated. When the device is closely wrapped around the body during use, the radiation focuses and penetrates approximately one inch into the body tissue, significantly amplifying its therapeutic effect.

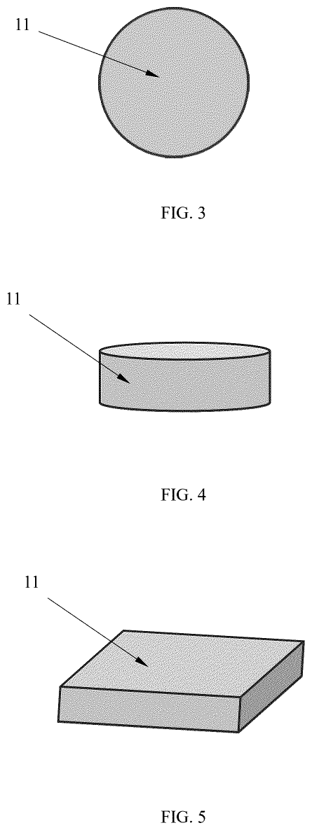

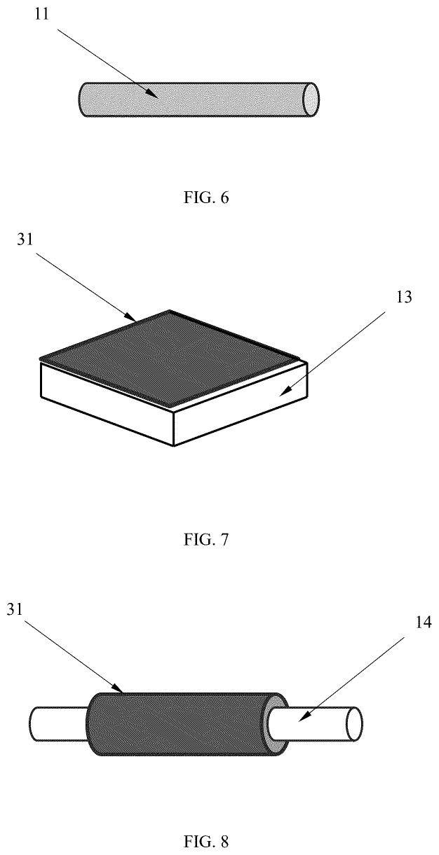

Nonetheless, the ceramic composites of the present invention can be formed into various shapes and sizes to suit different applications. In one embodiment, the ceramic composites are circular, with diameters ranging from 2 to 50 mm and a thickness of 1-10 mm. In another embodiment, the ceramic composites are rectangular, with dimensions ranging from 2×3 mm to 40×50 mm and a thickness of 1-10 mm. Circular and rectangular ceramics are generally easier to fabricate compared to other shapes. FIGS. 3-6 illustrate four separate embodiments of the present invention, in which the ceramic composite (11) takes the form of a sphere, a circular plate, a rectangular plate, and a rod or wire.

The ceramic composite described herein can also be ground, crushed, milled, or otherwise mechanically processed into submicron particles, preferably with a grain size greater than about 50 nanometers. These finely ground particles can be mixed into elastomer materials, including synthetic resin, polyvinyl chloride, silicone rubber, polyurethane, polystyrene, polyethylene, or similar substances, to create shaped articles. Smaller grain sizes may be beneficial for mixing with elastomer materials, as smaller grain sizes improve dispersion, increase contact surface area, ensure smooth finishing, and enhance performance. However, a particle size greater than about 50 nanometers may also be beneficial because particles smaller than about 50 nanometers may present challenges such as agglomeration, processing difficulties, higher costs, and potential health risks. The resulting articles may take forms such as foam, pads, sheets, mats, beads, or plates, with potential shapes including rectangular, circular, cylindrical, or spherical, as illustrated in FIGS. 3-6.

Mixing ceramic powders with elastomer materials involves several steps to ensure proper dispersion and compatibility. These steps include selecting and preparing the materials, applying a coupling agent to the ceramic powders, blending the powders into the elastomer matrix, maintaining an appropriate temperature during mixing, and cooling the mixture. When mixing with elastomer material, the weight percentage of ceramic powders can range from 5 % to 30 %, depending on the flexibility, cost, and effectiveness of the radiation, while 10 wt. % is preferred.

In another embodiment, ceramic composite particles (31) are deposited onto a base material (13)—including flexible materials like cloth, plastic, and rubber, as well as rigid materials like wood, ceramic, and metal—as illustrated in FIG. 7. Ceramic composite particles can be deposited or coated onto a base material through various methods, depending on the application. Common techniques include thermal spraying, the sol-gel process, electrophoretic deposition (EPD), chemical vapor deposition (CVD), and physical vapor deposition. Additionally, the ceramic composite particles (31) may be coated onto wires or fibers (14), as depicted in FIG. 8, to enhance their functionality. Coating ceramic composite particles onto fibers can be accomplished using various techniques, including EPD, CVD, the sol-gel process, spray coating, and dip coating. These devices can be equipped with flexible, semi-rigid, or rigid attachment mechanisms, such as straps, bands, tape, cords, belts or other bindings, for securely attaching the article to a body part undergoing treatment. The attachment mechanisms may use fasteners to attach and detach such devices from a body part, such as hook-and-loop fasteners, snaps, zip fasteners, magnets, clips, adhesives, or other removable fasteners.

The embodiments of the present invention described so far are passive devices. The ceramic composite absorbs ambient radiation heat to stimulate phonon-activated dynamic J-T distortions, enabling the emission of photons within the 3-16 μm wavelength range. The FIR-photon emission from the ceramic composite persists indefinitely as long as the ambient temperature remains above Absolute Zero (0 K or −273 °C). Naturally, body heat serves as an effective heat source for the ceramic composite to emit 3-16 μm FIR photons.

On the other hand, ionizing radiation from the ceramic composite persists for an extended period because it is derived from naturally occurring radioactive isotopes, which inherently have extremely long lifetimes. The radioactivity of an isotope is largely determined by its half-life. For example, Uranium-239 has a half-life of 4.5 billion years, while Thorium- 232 boasts an even longer half-life of 14 billion years, spanning the age of the Earth itself.

These features make the therapeutic device based on the ceramic composite of the present invention a simple, effective, user-friendly, and maintenance-free solution. The device operates without the need for an external power supply, relying solely on body heat.

Exploratory sample ceramic composites were developed, and promising results were observed in mouse models and clinical trials. These results were particularly notable in promoting wound healing and addressing neurodegenerative conditions, such as Parkinson's disease (PD) and Alzheimer's disease (AD).

### CONCLUSION, RAMIFICATIONS, AND SCOPE

According to the present invention, a ceramic composite for treating the human or animal body—based on the radiation hormesis theory—can simultaneously emit nonthermal far infrared photons within the 3-16 μm wavelength spectrum and ionizing radiation at a specific dose rate exceeding 0.05 μSv/h (micro-Sieverts per hour). This ceramic composite can be used on its own or as a component of a therapeutic device designed to enhance physiological performance, immune competence, overall health, and the mean lifespan of humans or animals.

The invention has been described above. It is evident that numerous modifications and variations of the present invention are possible in light of the above teachings. These variations should not be considered departures from the spirit and scope of the invention. All such modifications that would be apparent to those skilled in the art are intended to fall within the scope of the following claims.

1. A method of producing nonthermal far infrared and ionizing radiation emitting ceramic composite comprising:
a). providing a first set of powdered substance comprising at least 8 wt. % (weight percentage) silicon oxide (SiO₂) and at least 2 wt. % aluminum oxide (Al₂O₃);
b). providing a second set of powdered substance comprising at least 2 wt. % chromium oxide (Cr₂O₃) or iron oxide (Fe₂O₃);
c). providing a third powdered substance comprising at least 0.1 wt. % radioactive isotope;
d). mixing said first, second and third sets of powdered substances with bonding agent;
e). calcining the mixture of step d) through a predetermined process to form a ceramic composite,
wherein step e) produces transformation into a polycrystalline structure comprising at least a polymerized silicate structure with distorted octahedral compound, and
wherein as a result of calcination, the ceramic composite is capable of simultaneously emitting nonthermal far infrared radiation within 3-16 μm wavelength spectrum and ionizing radiation at a dose rate greater than 0.05 μSv/h.

2. The method of claim 1, further comprising:
f). grinding said ceramic composite into a powder and mixing said powder into one or more other compounds to form a module.

3. The method of claim 2, wherein:
the powder has a grain size of greater than about 50 nanometers in diameter.

4. The method of claim 2, wherein:
the powder is mixed with one or more compounds including synthetic resin, polyvinyl chloride, silicone rubber, polyurethane, polystyrene, or polyethylene.

5. The method of claim 1, further comprising:
f). grinding said ceramic composite into a powder and coating a substrate with said powder.

6. The method of claim 5, wherein:
the substrate is one of: cloth, plastic, rubber, wood, ceramic, or metal.

7. A method of claim 1, wherein:
the first set of powdered substance includes pure silicon oxide or aluminum oxide.

8. A method of claim 1, wherein:
the first set of powdered substance includes one or more minerals that contain silicon oxide or aluminum oxide.

9. A method of claim 1, wherein:
the first set of powdered substance includes one or more minerals selected from aluminosilicate group.

10. A method of claim 1, wherein:
the second set of powdered substance includes pure chromium oxide or iron oxide.

11. A method of claim 1, wherein:
the second set of powdered substance includes one or more minerals, such as hematite, magnetite, or chromite.

12. A method of claim 1, wherein:
the third set of powdered substance includes at least one radioactive isotope of uranium (U), thorium (Th), potassium (K), cobalt (Co), or radium (Ra).

13. A method of claim 1, wherein:
the third set of powdered substance includes at least one oxide that contains a radioactive isotope of uranium, thorium, potassium, cobalt, or radium.

14. A method of claim 1, wherein:
the third set of powdered substance includes a mineral that contains at least a radioactive isotope of uranium, thorium, potassium, cobalt, or radium.

15. A method of claim 14, wherein:
the mineral is thorium-rich monazite.

16. A method of claim 1, wherein:
the process includes calcining at a temperature at 1200 °C or higher for at least 2 hours.

17. A method of claim 1, wherein:
the polymerized silicate structure is a phyllosilicate structure.

18. A method of claim 17, wherein:
the phyllosilicate structure includes at least a tetrahedral sheet of silicate and an octahedral sheet with Al³⁺ ions in some of the octahedral sites, while the radioactive isotope is deposited in the interstitial layer.

19. A method of claim 1, wherein:
the distorted octahedral compound includes substitution of Fe³⁺ or Cr³⁺ ion for Al³⁺ ion in the octahedral site.

20. A composition of nonthermal far infrared and ionizing radiation emitting ceramic composite comprising:
at least 8 wt. % silicon oxide (SiO₂) and at least 2 wt. % aluminum oxide (Al₂O₃),
at least 2 wt. % chromium oxide (Cr₂O₃) or iron oxide (Fe₂O₃), and
at least 0.1 wt. % radioactive isotope,
wherein the mixture of said substances is calcined through a predetermined process to form a ceramic composite that at least consists of a polymerized silicate structure and includes a distorted octahedral compound, said ceramic composite being capable of simultaneously emitting nonthermal far infrared radiation within 3-16 μm wavelength spectrum and ionizing radiation at a dose rate greater than 0.05 μSv/h.

21. A therapeutic device for treating a human or animal body comprising:
a ceramic composite capable of emitting nonthermal far infrared radiation within 3-16 μm wavelength spectrum and ionizing radiation at a dose rate greater than 0.05 μSv/h affixed with a flexible means for attaching the composite to a body part to be treated.

22. A therapeutic device for treating a human or animal body comprising:
a ceramic composite capable of emitting nonthermal far infrared radiation within 3-16 μm wavelength spectrum and ionizing radiation at a dose rate greater than 0.05 μSv/h, said ceramic composite being ground into ceramic particles and applied to a material to form a shaped article.

23. The therapeutic device of claim 22, wherein:
the grain size of ceramic particles is greater than 50 nm (nanometers).

24. The therapeutic device of claim 22, wherein:
the ceramic particles are embedded in elastomer material, including synthetic resin, polyvinyl chloride, silicone rubber, polyurethane, polystyrene, or polyethylene, to form a shaped article in form of foam, pad, sheet, mat, bead, or plate.

25. The therapeutic device of claim 22, wherein:
the ceramic particles are deposited on base material.

26. The therapeutic device of claim 22, wherein:
the ceramic particles are coated onto fibers or cloth.

27. The therapeutic device of claim 22, wherein:
the shaped article is affixed with a flexible means for attaching the article to a body part to be treated.
